(19) United States
(12) Patent Application Publication
(10) Pub. No.: US 2026/0224199 A1
(43) Pub. Date: Aug. 6, 2026

(54) ULTRASOUND DIAGNOSTIC APPARATUS AND ULTRASOUND DIAGNOSTIC PROGRAM

(71) Applicant: FUJIFILM Corporation, Tokyo (JP)

(72) Inventor: Sho TANABE, Tokyo (JP)

(73) Assignee: FUJIFILM Corporation, Tokyo (JP)

(21) Appl. No.: 19/437,357

(22) Filed: Dec. 31, 2025

(30) Foreign Application Priority Data
Jan. 31, 2025 (JP) 2025-015210

(51) Int. Cl. A61B 8/00; G01N 29/30; G01N 29/44; G01N 29/24

(52) U.S. Cl. CPC A61B 8/4438; G01N 29/30; G01N 29/4454; G01N 29/4463; G01N 29/24

(57) ABSTRACT

A probe identification unit identifies a type of an ultrasound probe connected to a probe connector. The circuit control unit controls the reception circuit and changes at least a part of the circuit configuration of the reception circuit according to the type of the ultrasound probe identified by the probe identification unit. For example, the circuit control unit controls the AFE such that the AFE converts the analog reception signal into the digital reception signal at a sampling frequency corresponding to the type of the ultrasound probe. In addition, the circuit control unit changes a circuit configuration of a filter of the decimation block according to the type of the ultrasound probe.

### CROSS-REFERENCE TO RELATED APPLICATION

This application claims the priority benefit of Japan application serial no. 2025-015210, filed on January 31, 2025. The entirety of the above-mentioned patent application is hereby incorporated by reference herein and made a part of this specification.

### BACKGROUND OF THE INVENTION

### 1. Field of the Invention

The present specification discloses improvements in an ultrasound diagnostic apparatus and an ultrasound diagnostic program.

### 2. Description of the Related Art

In the related art, an ultrasound diagnostic apparatus configured to include an ultrasound probe and an apparatus main body is known. The ultrasound probe transmits an ultrasound wave toward a subject based on a transmission signal sent from the apparatus main body, receives a reflected wave of the ultrasound wave from the subject, and transmits a reception signal, which is an electric signal based on the reflected wave, to the apparatus main body. The apparatus main body forms an ultrasound image based on the reception signal. Examples of the ultrasound image include an ultrasound tomographic image (B-mode image) representing a cross section in the subject, an M-mode image representing movement of a tissue (for example, a heart valve or myocardium) in the subject, and a Doppler image representing a flow velocity and a flow direction of blood flow in the subject.

There are various types of ultrasound probes. In the apparatus main body, it is preferable to perform processing of a content corresponding to the type of the ultrasound probe. Therefore, in the related art, an ultrasound diagnostic apparatus that performs processing corresponding to the type of the ultrasound probe has been proposed.

For example, JP1993-253220A (JP-H5-253220A) discloses an ultrasound diagnostic apparatus that detects a signal representing a type of an ultrasound probe connected to an apparatus main body, selects a quality adjustment signal corresponding to the type of the ultrasound probe, and performs quality adjustment processing of an ultrasound image using the selected quality adjustment signal. In addition, JP2009-279023A discloses an ultrasound diagnostic apparatus that holds a type of an ultrasound probe connected to an apparatus main body and latest transmission/reception conditions (including a transmission frequency) in association with each other, acquires a type of the ultrasound probe after being replaced in a case where the ultrasound probe is replaced, and maintains the held transmission/reception conditions in a case where the type of the ultrasound probe after being replaced is the same as the type of the ultrasound probe before being replaced.

### SUMMARY OF THE INVENTION

In some cases, a plurality of types of ultrasound probes can be connected to the apparatus main body. In that case, an appropriate reception circuit corresponding to the type of the ultrasound probe may be required on the apparatus main body side to process the reception signal from the ultrasound probe.

Here, it is considered to provide a plurality of reception circuits suitable for each of the plurality of types of ultrasound probes in the apparatus main body. However, in this method, a problem may occur in which a cost of the apparatus main body increases. In addition, it is considered to provide a high-performance reception circuit that can process the reception signal from all ultrasound probes that can be connected to the apparatus main body in the apparatus main body. However, in this method, the reception circuit may be over-specified, and a problem may occur in which unnecessary heat generation or power consumption occurs in the reception circuit.

An object of the ultrasound diagnostic apparatus disclosed in the present specification is to appropriately process the reception signal from each ultrasound probe in the apparatus main body while suppressing an increase in cost and occurrence of unnecessary heat generation or power consumption in a case where a plurality of types of ultrasound probes can be connected to the apparatus main body.

According to the present specification, an ultrasound diagnostic apparatus comprises an ultrasound probe that transmits an ultrasound wave to a subject and receives a reflection of the ultrasound wave from the subject to output an analog reception signal, a reception circuit that processes the analog reception signal, and a processor, and the processor is configured to: identify a type of the ultrasound probe; control the reception circuit according to the type of the ultrasound probe; and change at least a part of a circuit configuration of the reception circuit.

The reception circuit may include an AD converter that converts the analog reception signal into a digital reception signal and a processing circuit that processes the digital reception signal, and the processor may be configured to: control the AD converter such that the AD converter converts the analog reception signal into the digital reception signal at a sampling frequency corresponding to the type of the ultrasound probe; and change a circuit configuration of the processing circuit according to the type of the ultrasound probe.

The processing circuit may execute decimation processing of the digital reception signal.

The processor may be configured to change a circuit configuration of a filter for the decimation processing according to the type of the ultrasound probe.

The processor may be configured to: control the AD converter such that the AD converter converts the analog reception signal into the digital reception signal at a higher sampling frequency as a frequency of the analog reception signal output by the ultrasound probe increases; and reduce the number of filters as the frequency of the analog reception signal output by the ultrasound probe increases.

The processing circuit may be implemented by an FPGA.

The processor may be configured to change a part of a circuit configuration of the FPGA.

The processor may be configured to notify an operator of the ultrasound diagnostic apparatus whether the change of the circuit configuration of the reception circuit is successful or unsuccessful.

In addition, according to the present specification, an ultrasound diagnostic program causes an ultrasound diagnostic apparatus to execute: identifying a type of an ultrasound probe that is connected to the ultrasound diagnostic apparatus, and that transmits an ultrasound wave to a subject and receives a reflection of the ultrasound wave from the subject to output an analog reception signal; and controlling a reception circuit that processes the analog reception signal according to the type of the ultrasound probe and changing at least a part of a circuit configuration of the reception circuit.

According to the ultrasound diagnostic apparatus disclosed in the present specification, in a case where a plurality of types of ultrasound probes can be connected to an apparatus main body, the reception signal from each ultrasound probe can be appropriately processed in the apparatus main body while suppressing an increase in cost and occurrence of unnecessary heat generation or power consumption.

### BRIEF DESCRIPTION OF THE DRAWINGS

FIG. 1 is a schematic diagram of a configuration of an ultrasound diagnostic apparatus according to the present embodiment.

FIG. 2 is a schematic diagram of a configuration of a reception unit and a processor.

FIG. 3 is a conceptual diagram showing a filter formed in a decimation block in a case where the ultrasound probe is a general-purpose probe.

FIG. 4 is a conceptual diagram showing a filter formed in the decimation block in a case where the ultrasound probe is a high-frequency probe.

FIG. 5 is a flowchart showing a flow of processing of the ultrasound diagnostic apparatus according to the present embodiment.

### DESCRIPTION OF THE PREFERRED EMBODIMENTS

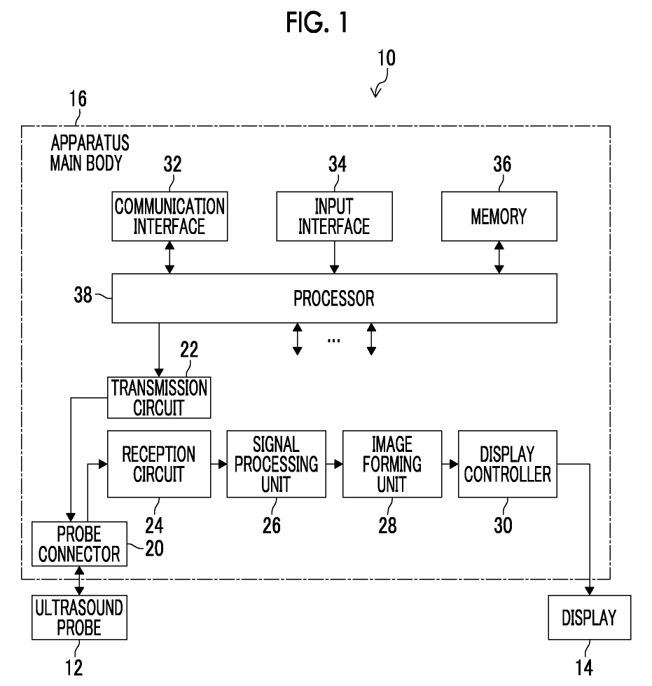

FIG. 1 is a schematic diagram of a configuration of an ultrasound diagnostic apparatus 10 according to the present embodiment. The ultrasound diagnostic apparatus 10 is a medical apparatus installed in medical institutions, such as a hospital. The ultrasound diagnostic apparatus 10 includes an ultrasound probe 12, a display 14, and an apparatus main body 16.

The ultrasound probe 12 is a device that transmits and receives an ultrasound wave to and from the subject. The ultrasound probe 12 includes a transducer element array consisting of a plurality of transducer elements that perform scanning of an ultrasound beam to the subject. A transmission signal is supplied from the apparatus main body 16 (specifically, a transmission circuit 22 described below) to each transducer element, so that each transducer element transmits an ultrasound wave to the subject. In addition, each transducer element receives a reflected wave of the transmitted ultrasound wave from the subject. The ultrasound probe 12 forms a reception signal based on the received reflected wave and outputs the reception signal to the apparatus main body 16. In the present embodiment, the ultrasound probe 12 transmits an analog reception signal, which is an analog electric signal, to the apparatus main body 16.

Although details will be described below, in the ultrasound diagnostic apparatus 10, a plurality of types of ultrasound probes 12 can be connected to the apparatus main body 16. In the present embodiment, the ultrasound probe 12 is attachably and detachably connected to the apparatus main body 16 (specifically, a probe connector 20 described below). An operator of the ultrasound diagnostic apparatus 10 selects an appropriate type of the ultrasound probe 12 according to a purpose and connects the ultrasound probe 12 to the apparatus main body 16.

The display 14 as a display unit is a display device configured by, for example, a liquid crystal display or an organic electroluminescence (EL). The display 14 is connected to the apparatus main body 16. The display 14 displays various types of information in addition to the ultrasound image formed in the apparatus main body 16 based on the reception signal output by the ultrasound probe 12 in response to an instruction from the apparatus main body 16 (specifically, a display controller 30 described below).

Hereinafter, the apparatus main body 16 will be described.

The probe connector 20 is a connector for connecting the ultrasound probe 12. A plurality of types of ultrasound probes 12 can be connected to the probe connector 20. In the present embodiment, a plurality of types of ultrasound probes 12 that output analog reception signals having different frequencies can be connected to the probe connector 20.

For example, a transmission frequency that can be transmitted by the ultrasound probe 12 is determined according to the transducer element or a peripheral member (for example, a backing member) of the ultrasound probe 12. The ultrasound probe 12 having a high transmission frequency can form a high-resolution and high-quality ultrasound image, but it is difficult to image a deep part of the subject because the ultrasound wave is easily attenuated. On the other hand, the ultrasound probe 12 having a low transmission frequency can image a deep part of the subject because the ultrasound wave is not easily attenuated, but the resolution of the ultrasound image is relatively low.

A frequency of the reflected wave from the subject corresponds to the transmission frequency, and a frequency of the analog reception signal output by the ultrasound probe 12 also corresponds to the transmission frequency. That is, the ultrasound probe 12 that transmits an ultrasound wave having a high transmission frequency outputs an analog reception signal having a high frequency, and the ultrasound probe 12 that transmits an ultrasound wave having a low transmission frequency outputs an analog reception signal having a low frequency.

In the present specification, the ultrasound probe 12 that outputs an analog reception signal having a high frequency (for example, 30 MHz or more) is referred to as a "high-frequency probe", and the ultrasound probe 12 that outputs an analog reception signal having a low frequency (for example, less than 20 MHz) is referred to as a "general-purpose probe". It should be noted that three or more types of ultrasound probes 12 that output analog reception signals having three or more different frequencies may be connectable to the probe connector 20.

The transmission circuit 22 transmits the transmission signal to the ultrasound probe 12 (specifically, each transducer element of the transducer element array) in response to an instruction from a processor 38 described below. As a result, the ultrasound probe 12 transmits the ultrasound wave to the subject. The transmission circuit 22 transmits the transmission signal for transmitting the ultrasound wave having a high transmission frequency (for example, 30 MHz or more) to the high-frequency probe, and transmits the transmission signal for transmitting the ultrasound wave having a low transmission frequency (for example, less than 20 MHz) to the general-purpose probe. It should be noted that, as will be described below, the type of the ultrasound probe 12 connected to the probe connector 20 (whether the ultrasound probe 12 is the high-frequency probe or the general-purpose probe) is identified by the processor 38.

The reception circuit 24 receives the analog reception signal from the ultrasound probe 12. The reception circuit 24 performs processing of converting the received analog reception signal into a digital reception signal, phase alignment and addition processing (beam forming processing) of adding reception signals from each transducer element by aligning phases, and the like. As a result, a reception beam signal is formed in which information indicating the signal intensity of reflected waves from the subject is arranged in a depth direction of the subject.

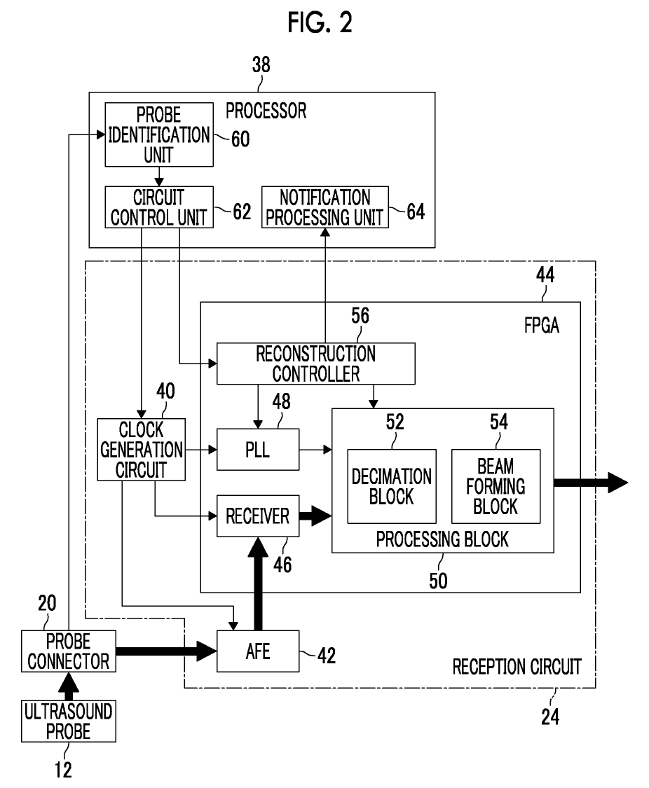

In the present embodiment, a circuit configuration of at least a part of a circuit included in the reception circuit 24 can be dynamically changed (that is, without restarting the ultrasound diagnostic apparatus 10 or the like). For example, in the present embodiment, at least a part of the circuit included in the reception circuit 24 can transition between a configuration capable of executing processing suitable for the reception signal from the high-frequency probe and a configuration capable of executing processing suitable for the reception signal from the general-purpose probe. Details of the reception circuit 24 will be described below with reference to FIGS. 2 to 5.

A signal processing unit 26 performs various types of signal processing including filter processing of applying a bandpass filter, detection processing, and the like, on the reception beam signal from the reception circuit 24. In addition, the signal processing unit 26 may obtain a Doppler signal indicating a flow rate and a flow direction of blood flow in the subject by executing quadrature detection processing, autocorrelation calculation, or the like on the reception beam signal.

The image forming unit 28 forms the ultrasound image based on the reception beam signal subjected to the signal processing in the signal processing unit 26. The ultrasound image may include a B-mode image, an M-mode image, a Doppler image, and the like.

The display controller 30 performs control to display various images including the ultrasound image formed by the image forming unit 28 on a display 14.

A communication interface 32 is configured by, for example, a network adapter. The communication interface 32 exhibits a function of communicating with other devices via the communication line.

An input interface 34 is configured by, for example, a button, a trackball, or a touch panel. The input interface 34 is used to input an instruction of an operator who uses the ultrasound diagnostic apparatus 10 to the ultrasound diagnostic apparatus 10.

A memory 36 includes a hard disk drive (HDD), a solid state drive (SSD), an embedded Multi Media Card (eMMC), a read only memory (ROM), a random access memory (RAM), or the like. The memory 36 stores the ultrasound diagnostic program for operating each unit of the ultrasound diagnostic apparatus 10. The ultrasound diagnostic program can also be stored in, for example, a computer-readable non-transitory storage medium such as a universal serial bus (USB) memory or a CD-ROM. The ultrasound diagnostic apparatus 10 can read the ultrasound diagnostic program from such a storage medium and execute the ultrasound diagnostic program.

The processor 38 is configured to include a central processing unit (CPU), for example. The processor 38 controls each unit of the apparatus main body 16 by the ultrasound diagnostic program stored in the memory 36. Details of functions exhibited by the processor 38 will be described below with reference to FIGS. 2 to 5.

FIG. 2 is a schematic diagram of a configuration of a reception circuit 24 and a processor 38. A bold arrow extending from the ultrasound probe 12 → the probe connector 20 → an AFE 42 → a receiver 46 → a processing block 50 in FIG. 2 indicates a path of the reception signal. First, a configuration of the reception circuit 24 will be described, and an operation of the reception circuit 24 will be described below together with functions of the processor 38.

The reception circuit 24 includes a clock generation circuit 40, an analog front-end (AFE) 42, and a field programmable gate array (FPGA) 44.

The clock generation circuit 40 includes a clock IC or the like. The clock generation circuit 40 supplies a clock signal to the AFE 42 and the FPGA 44 (particularly, the receiver 46 and a Phase Locked Loop (PLL) 48 described below) in response to an instruction from the processor 38 (specifically, a circuit control unit 62 described below). In the present embodiment, the clock generation circuit 40 can change a frequency (clock frequency) of the clock signal to be supplied to the AFE 42 and the FPGA 44 in response to an instruction from the circuit control unit 62.

The AFE 42 converts the analog reception signal received from the ultrasound probe 12 via the probe connector 20 into the digital reception signal. That is, the AFE 42 functions as an AD converter. The AFE 42 converts the analog reception signal into the digital reception signal by sampling the analog reception signal at a sampling frequency corresponding to the frequency of the clock signal supplied from the clock generation circuit 40.

The FPGA 44 is a device in which gates (logic circuits) capable of programming a configuration of a logic circuit by a designer of the ultrasound diagnostic apparatus 10 in the field are integrated. In the present embodiment, the FPGA 44 is a processing circuit that processes the digital reception signal from the AFE 42 and outputs the processed signal to the signal processing unit 26 (see FIG. 1). The FPGA 44 exhibits functions of the receiver 46, a phase locked loop (PLL) 48, the processing block 50 (including a decimation block 52 and a beam forming block 54), and a reconstruction controller 56. Although details will be described below, at least a part of the circuit configuration of the FPGA 44 is dynamically changed in response to an instruction from the processor 38 (specifically, the circuit control unit 62 described below). In the present embodiment, the circuit configuration of the decimation block 52 included in the processing block 50 is dynamically changed in response to an instruction from the processor 38.

The receiver 46 is a serial interface for connecting the AFE 42 and the processing block 50. The receiver 46 receives the digital reception signal from the AFE 42 and transmits the digital reception signal to the processing block 50. The receiver 46 operates based on the clock signal supplied from the clock generation circuit 40.

The PLL 48 is a circuit that generates a stable clock signal based on the clock signal input from the clock generation circuit 40. The PLL 48 can output a frequency-doubled clock signal of the input clock signal or a clock signal having any frequency. The clock signal from the PLL 48 is supplied to the processing block 50. The PLL 48 changes the frequency of the output clock signal in response to an instruction from the reconstruction controller 56 described below.

The processing block 50 is a circuit that operates based on the clock signal from the PLL 48 and performs processing on the digital reception signal from the receiver 46. In the present embodiment, the processing block 50 includes the decimation block 52 and the beam forming block 54.

The decimation block 52 executes decimation processing of the digital reception signal. By reducing the amount of data of the digital reception signal by the decimation processing, a processing load on each unit that performs processing on the digital reception signal after that is reduced. In the decimation block 52, a plurality of types of filters are formed, and the decimation processing is executed by applying a filter selected by the user to the reception signal.

The beam forming block 54 performs phase alignment and addition processing of adding the plurality of reception signals from each transducer element by aligning the phases. As a result, the reception beam signal is formed. It should be noted that, in the present embodiment, the beam forming block 54 performs the phase alignment and addition processing on the digital reception signal subjected to the decimation processing by the decimation block 52, but the decimation processing by the decimation block 52 may be executed after the phase alignment and addition processing by the beam forming block 54.

The reconstruction controller 56 controls the PLL 48 and the processing block 50 in response to an instruction from the processor 38 (specifically, the circuit control unit 62 described below). Specifically, the reconstruction controller 56 controls the frequency of the clock signal output from the PLL 48 and dynamically changes the circuit configuration of the processing block 50. In the present embodiment, the reconstruction controller 56 dynamically changes the circuit configuration of the decimation block 52. Details of the processing performed by the reconstruction controller 56 will be described below together with the processing performed by the circuit control unit 62.

Hereinafter, the operation of the reception circuit 24 will be described together with the functions exhibited by the processor 38.

A probe identification unit 60 identifies the type of the ultrasound probe 12 connected to the probe connector 20. Specifically, a type ID (for example, a model number) for identifying the type of the ultrasound probe 12 is stored in a memory in the ultrasound probe 12, and the circuit control unit 62 acquires the type ID from the ultrasound probe 12 via the probe connector 20. The probe identification unit 60 identifies the type of the ultrasound probe 12 connected to the probe connector 20 based on the type ID.

In the present embodiment, the probe identification unit 60 identifies the frequency of the reception signal output by the ultrasound probe 12 connected to the probe connector 20 by referring to probe information in which the type of the ultrasound probe 12 and the frequency of the reception signal output by the ultrasound probe 12 of the type are associated with each other. It should be noted that the probe information may be stored in the memory 36 or may be stored in another device (for example, a server) that can communicate with the communication interface 32.

The circuit control unit 62 controls the reception circuit 24 and changes at least a part of the circuit configuration of the reception circuit 24 according to the type of the ultrasound probe 12 identified by the probe identification unit 60. Specifically, the circuit control unit 62 specifies a control method of the reception circuit 24 and a circuit configuration of the reception circuit 24 suitable for the type of the ultrasound probe 12 identified by the probe identification unit 60 by referring to circuit control information in which the type of the ultrasound probe 12 and the control method of the reception circuit 24 and the circuit configuration of the reception circuit 24 are associated with each other. The circuit control information may be stored in the memory 36 or may be stored in another device (for example, a server) that can communicate with the communication interface 32.

In particular, in the present embodiment, since the probe identification unit 60 identifies whether the ultrasound probe 12 connected to the probe connector 20 is the general-purpose probe or the high-frequency probe, the circuit control unit 62 controls the reception circuit 24 such that the reception circuit 24 operates differently in a case where the ultrasound probe 12 is the general-purpose probe and in a case where the ultrasound probe 12 is the high-frequency probe. In addition, the circuit control unit 62 changes at least a part of the circuit configuration of the reception circuit 24 such that at least a part of the circuit configuration of the reception circuit 24 is different between the case where the ultrasound probe 12 is the general-purpose probe and the case where the ultrasound probe 12 is the high-frequency probe. Hereinafter, the description thereof will be made in detail.

First, the circuit control unit 62 controls the clock generation circuit 40 to output a frequency corresponding to the type of the ultrasound probe 12. As described above, the AFE 42 converts the analog reception signal into the digital reception signal at a sampling frequency corresponding to the frequency of the clock signal supplied from the clock generation circuit 40. That is, it can be said that the circuit control unit 62 controls the AFE 42 such that the AFE 42 converts the analog reception signal into the digital reception signal at a sampling frequency corresponding to the type of the ultrasound probe 12 via the clock generation circuit 40.

Specifically, the circuit control unit 62 controls the AFE 42 such that the AFE 42 converts the analog reception signal into the digital reception signal at a high sampling frequency by causing the clock generation circuit 40 to output a high-frequency clock signal to the AFE 42 as the frequency of the analog reception signal output by the ultrasound probe 12 increases. In other words, the circuit control unit 62 controls the AFE 42 such that the AFE 42 converts the analog reception signal into the digital reception signal at a low sampling frequency by causing the clock generation circuit 40 to output a low-frequency clock signal to the AFE 42 as the frequency of the analog reception signal output by the ultrasound probe 12 decreases.

In the present embodiment, in a case where the ultrasound probe 12 is the general-purpose probe, the circuit control unit 62 controls the clock generation circuit 40 to supply the AFE 42 with a low-frequency clock signal of a frequency low enough to faithfully convert the analog reception signal having a low frequency output by the general-purpose probe into the digital reception signal. According to the sampling theorem, in order to faithfully convert the analog reception signal into the digital reception signal, the analog reception signal needs to be sampled at a sampling frequency of twice or more the analog reception signal. Therefore, for example, in a case where the frequency of the analog reception signal output by the general-purpose probe is 20 MHz, the circuit control unit 62 controls the clock generation circuit 40 to supply the AFE 42 with a clock signal having a frequency of 40 MHz.

On the other hand, in a case where the ultrasound probe 12 is the high-frequency probe, the circuit control unit 62 controls the clock generation circuit 40 to supply the AFE 42 with a high-frequency clock signal of a frequency high enough to faithfully convert the analog reception signal having a high frequency output by the high-frequency probe into the digital reception signal. For example, in a case where the frequency of the analog reception signal output by the high-frequency probe is 30 MHz, the circuit control unit 62 controls the clock generation circuit 40 to supply the AFE 42 with a clock signal having a frequency of 60 MHz.

As described above, in a case where the ultrasound probe 12 is the general-purpose probe, the AFE 42 can convert the analog reception signal into the digital reception signal at a low sampling frequency, so that unnecessary heat generation or power consumption in the AFE 42 can be suppressed. In addition, the data capacity of the digital reception signal can be prevented from being unnecessarily large, and an unnecessary load on the subsequent circuit can be suppressed. On the other hand, in a case where the ultrasound probe 12 is the high-frequency probe, the AFE 42 can convert the analog reception signal into the digital reception signal at a high sampling frequency, so that the high-frequency analog reception signal can be faithfully converted into the digital signal.

In a case where the AFE 42 converts the analog reception signal into the digital reception signal at a high sampling frequency, the data capacity of the digital reception signal increases. Therefore, the circuit control unit 62 controls the clock generation circuit 40 to supply the receiver 46 with the clock signal having a frequency corresponding to the type of the ultrasound probe 12 such that the receiver 46 can appropriately process the digital reception signal received from the AFE 42.

In addition, in the present embodiment, in a case where the ultrasound probe 12 is the general-purpose probe, the circuit control unit 62 controls the clock generation circuit 40 such that the clock generation circuit 40 outputs the low-frequency (for example, 13.3 MHz) clock signal to the receiver 46. On the other hand, in a case where the ultrasound probe 12 is the high-frequency probe, the circuit control unit 62 controls the clock generation circuit 40 such that the clock generation circuit 40 outputs the high-frequency (for example, 20 MHz) clock signal to the receiver 46.

As a result, in a case where the ultrasound probe 12 is the general-purpose probe, the unnecessary heat generation or power consumption in the receiver 46 can be suppressed by reducing the operation frequency of the receiver 46. On the other hand, in a case where the ultrasound probe 12 is the high-frequency probe, the receiver 46 can appropriately process the digital reception signal having a large data capacity by increasing the operation frequency of the receiver 46.

In addition, in the present embodiment, in a case where the ultrasound probe 12 is the general-purpose probe, the circuit control unit 62 causes the PLL 48 to supply the 40 MHz clock signal to the processing block 50 such that the processing block 50 can appropriately process the digital reception signal sampled at a sampling frequency of 40 MHz. As described above, the reconstruction controller 56 included in the FPGA 44 directly controls the PLL 48, but the circuit control unit 62 changes the frequency of the clock signal output from the PLL 48 to the reconstruction controller 56 by controlling the reconstruction controller 56. That is, it can be said that the circuit control unit 62 controls the PLL 48 via the reconstruction controller 56.

On the other hand, in a case where the ultrasound probe 12 is the high-frequency probe, the circuit control unit 62 causes the PLL 48 to supply the 60 MHz clock signal to the processing block 50 such that the processing block 50 can appropriately process the digital reception signal sampled at a sampling frequency of 60 MHz.

As a result, in a case where the ultrasound probe 12 is the general-purpose probe, the unnecessary heat generation or power consumption in the processing block 50 can be suppressed by reducing the operation frequency of the processing block 50. On the other hand, in a case where the ultrasound probe 12 is the high-frequency probe, the processing block 50 can appropriately process the digital reception signal sampled at a high sampling frequency by increasing the operation frequency of the processing block 50.

Further, the circuit control unit 62 changes a part of the circuit configuration of the FPGA 44 according to the type of the ultrasound probe 12 identified by the probe identification unit 60. In the present embodiment, the circuit control unit 62 changes the circuit configuration of the decimation block 52 in the processing block 50 according to the type of the ultrasound probe 12. As described above, the reconstruction controller 56 included in the FPGA 44 directly changes the circuit configuration of the decimation block 52, but the circuit control unit 62 changes the circuit configuration of the decimation block 52 to the reconstruction controller 56 by controlling the reconstruction controller 56. That is, it can be said that the circuit control unit 62 changes the circuit configuration of the decimation block 52 via the reconstruction controller 56.

As described above, a plurality of types of filters are formed in the decimation block 52, but in the present embodiment, the circuit control unit 62 changes the circuit configuration of the filter of the decimation block 52 according to the type of the ultrasound probe 12.

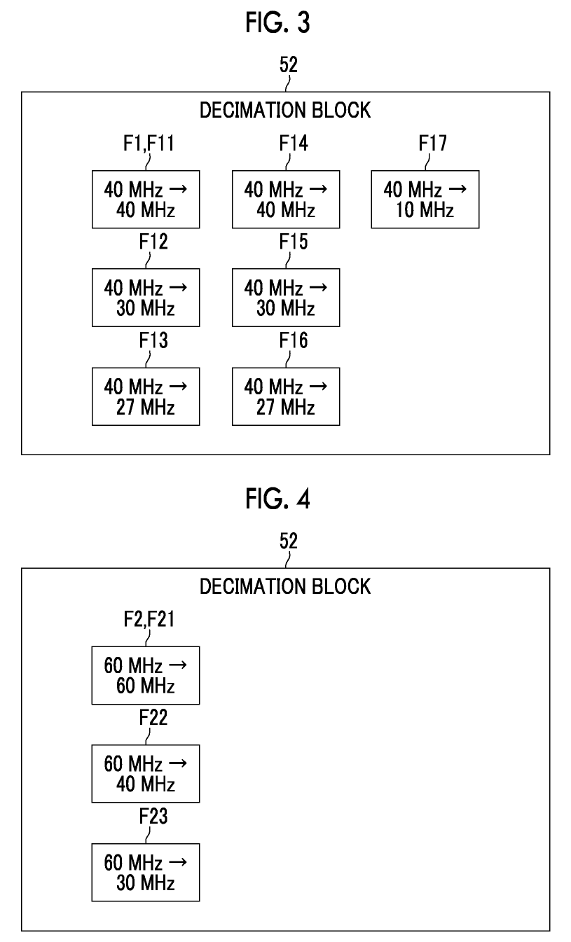

FIG. 3 is a conceptual diagram showing a filter F1 formed in the decimation block 52 in a case where the ultrasound probe 12 is the general-purpose probe. As shown in FIG. 3, in a case where the ultrasound probe 12 is the general-purpose probe, the circuit control unit 62 forms seven filters F1 of filters F11 to F17 in the decimation block 52. Then, the decimation processing is performed by passing the digital reception signal through the filter F1 selected by the operator.

Here, it is assumed that the analog reception signal from the general-purpose probe is sampled at a sampling frequency of 40 MHz by the AFE 42 and is converted into the digital reception signal. Each filter F1 samples the digital reception signal obtained at a sampling frequency of 40 MHz at a sampling frequency equal to or lower than the sampling frequency. For example, the filter F12 (40 MHz → 30 MHz) is a filter that generates a digital reception signal sampled at a sampling frequency of 30 MHz by decimating the sampling point from the digital reception signal having the sampling point of 40 MHz. In addition, the filter F13 (40 MHz → 27 MHz) is a filter that generates a digital reception signal sampled at a sampling frequency of 27 MHz by decimating the sampling point from the digital reception signal having the sampling point of 40 MHz. Needless to say, as the sampling frequency after passing through the filter F1 is lower, the accuracy of the digital reception signal is lower, but the data capacity of the digital reception signal is smaller. That is, the data capacity of the digital reception signal after passing through the filter F13 is smaller than the data capacity of the digital reception signal after passing through the filter F12.

It should be noted that the filter F11 (40 MHz → 40 MHz) is a filter that does not perform the decimation processing. The operator may select the filter F11 in a case where the decimation processing is not desired.

As shown in FIG. 3, by forming the plurality of filters F1, the operator can select any filter F1.

FIG. 4 is a conceptual diagram showing a filter F2 formed in the decimation block 52 in a case where the ultrasound probe 12 is the high-frequency probe. As shown in FIG. 4, in a case where the ultrasound probe 12 is the high-frequency probe, the circuit control unit 62 forms three filters F2 of filters F21 to F23 in the decimation block 52. Then, the decimation processing is performed by passing the digital reception signal through the filter F2 selected by the operator.

Here, it is assumed that the analog reception signal from the high-frequency probe is sampled at a sampling frequency of 60 MHz by the AFE 42 and is converted into the digital reception signal. Each filter F2 samples the digital reception signal obtained at a sampling frequency of 60 MHz at a sampling frequency equal to or lower than the sampling frequency. For example, the filter F22 (60 MHz → 40 MHz) is a filter that generates a digital reception signal sampled at a sampling frequency of 40 MHz by decimating the sampling point from the digital reception signal having the sampling point of 60 MHz. In addition, the filter F23 (60 MHz → 30 MHz) is a filter that generates a digital reception signal sampled at a sampling frequency of 30 MHz by decimating the sampling point from the digital reception signal having the sampling point of 60 MHz.

It should be noted that the filter F21 (60 MHz → 60 MHz) is a filter that does not perform the decimation processing. The operator may select the filter F21 in a case where the decimation processing is not desired.

As shown in FIG. 4, by forming the plurality of filters F2, the operator can select any filter F2.

As described above, the circuit configuration of the decimation block 52 is completely different between the case where the ultrasound probe 12 is the general-purpose probe and the case where the ultrasound probe 12 is the high-frequency probe. Specifically, the filter F1 formed in the decimation block 52 in a case where the ultrasound probe 12 is the general-purpose probe and the filter F2 formed in the decimation block 52 in a case where the ultrasound probe 12 is the high-frequency probe are completely different filters. The filter F1 is a filter suitable for the digital reception signal derived from the general-purpose probe, and the filter F2 is a filter suitable for the digital reception signal derived from the high-frequency probe. In a case where the filter F2 is applied to the digital reception signal derived from the general-purpose probe, the filter F2 is a filter for the digital reception signal formed at a high sampling frequency (60 MHz in the above example), so that an appropriate decimation processing result cannot be obtained. In a case where the filter F1 is applied to the digital reception signal derived from the high-frequency probe, the filter F1 is a filter for the digital reception signal formed at a low sampling frequency (40 MHz in the above example), so that an appropriate decimation processing result cannot be obtained. That is, the circuit control unit 62 changes the circuit configuration of the decimating filter of the decimation block 52 according to the type of the ultrasound probe 12 such that the digital reception signal derived from the ultrasound probe 12 can be appropriately processed.

In addition, the circuit control unit 62 may reduce the number of filters of the decimation block 52 as the frequency of the analog reception signal output by the ultrasound probe 12 increases. Even in the above example, in a case where the ultrasound probe 12 is the general-purpose probe, the circuit control unit 62 forms seven filters F1 of the decimation block 52, whereas in a case where the ultrasound probe 12 is the high-frequency probe, the circuit control unit 62 forms three filters F2 of the decimation block 52. This is because, in a case where the high-frequency probe is used, since it is difficult to image a deep part of the subject, the operator needs fewer types of filters.

Returning to FIG. 2, the notification processing unit 64 notifies the operator of the ultrasound diagnostic apparatus 10 whether the change of the circuit configuration of the reception circuit 24 is successful or unsuccessful under the control of the circuit control unit 62. In the present embodiment, the reconstruction controller 56 determines whether or not the reconstruction of the decimation block 52 is completed. For example, the reconstruction controller 56 can determine whether or not the reconstruction of the decimation block 52 is completed by receiving the reconstruction completion notification from the decimation block 52. In a case where the reconstruction of the decimation block 52 is completed, the reconstruction controller 56 outputs the reconstruction completion notification to the notification processing unit 64. In a case where the reconstruction completion notification is received from the reconstruction controller 56, the notification processing unit 64 outputs the notification to the operator.

Although the time is short, it takes time for the reconstruction controller 56 to change the circuit configuration of the decimation block 52 in response to the instruction of the circuit control unit 62, and the processing in the decimation block 52 cannot be performed while the circuit configuration of the decimation block 52 is being changed. In addition, it is also considered that the change processing of the circuit configuration of the decimation block 52 may fail. Therefore, by notifying the operator whether the change of the circuit configuration of the decimation block 52 is successful or unsuccessful, the operator can easily understand that the processing in the decimation block 52 can be performed.

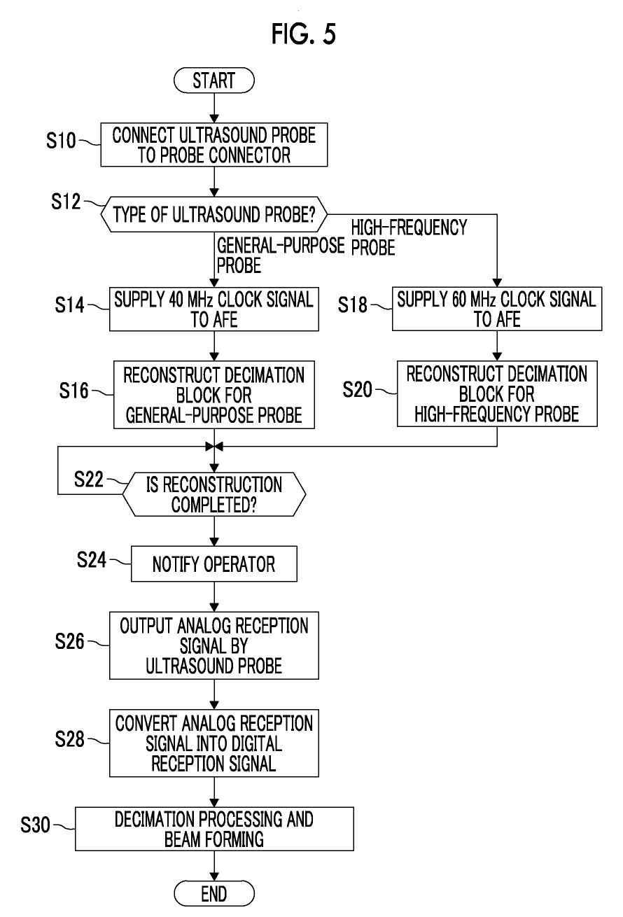

The schematic configuration of the ultrasound diagnosis apparatus 10 according to the present embodiment is as described above. Hereinafter, a flow of processing of the ultrasound diagnostic apparatus 10 will be described with reference to FIG. 5, which is a flowchart showing a flow of processing of the ultrasound diagnostic apparatus 10 according to the present embodiment.

In step S10, the ultrasound probe 12 is connected to the probe connector 20.

In step S12, the probe identification unit 60 identifies the type of the ultrasound probe 12. In a case where it is determined that the ultrasound probe 12 is the general-purpose probe, the processing proceeds to step S14.

In step S14, the circuit control unit 62 controls the clock generation circuit 40 to supply the 40 MHz clock signal to the AFE 42. In addition, the circuit control unit 62 controls the clock generation circuit 40 to supply the 13.3 MHz clock signal to the receiver 46. Further, the circuit control unit 62 controls the PLL 48 to supply the 40 MHz clock signal to the processing block 50.

In step S16, the circuit control unit 62 reconstructs the decimation block 52 and forms the seven filters F1 (see FIG. 3) of filters F11 to F17 in the decimation block 52. It should be noted that, in a case where the filters F11 to F17 are already formed in the decimation block 52, the decimation block 52 does not need to be reconstructed again.

In a case where it is determined that the ultrasound probe 12 is the high-frequency probe in step S12, the processing proceeds to step S18.

In step S18, the circuit control unit 62 controls the clock generation circuit 40 to supply the 60 MHz clock signal to the AFE 42. In addition, the circuit control unit 62 controls the clock generation circuit 40 to supply the 20 MHz clock signal to the receiver 46. Further, the circuit control unit 62 controls the PLL 48 to supply the 60 MHz clock signal to the processing block 50.

In step S20, the circuit control unit 62 reconstructs the decimation block 52 and forms the three filters F2 (see FIG. 4) of filters F21 to F23 in the decimation block 52. It should be noted that, in a case where the filters F21 to F23 are already formed in the decimation block 52, the decimation block 52 does not need to be reconstructed again.

In step S22, the reconstruction controller 56 determines whether or not the reconstruction of the decimation block 52 is completed. The reconstruction controller 56 waits until the reconstruction of the decimation block 52 is completed, and proceeds to step S24 in a case where it is determined that the reconstruction of the decimation block 52 is completed.

In step S24, the reconstruction controller 56 outputs the reconstruction completion notification to the notification processing unit 64. The notification processing unit 64 outputs the notification to the operator based on the reconstruction completion notification from the reconstruction controller 56.

In step S26, the ultrasound probe 12 transmits the ultrasound wave to the subject and receives the reflected wave from the subject. Then, the analog reception signal based on the reflected wave is output to the apparatus main body 16.

In step S28, the AFE 42 converts the analog reception signal from the ultrasound probe 12 into the digital reception signal by sampling the analog reception signal at a sampling frequency corresponding to the frequency of the clock signal supplied in step S14 or S18.

In step S30, the decimation block 52 reconstructed in step S16 or S20 executes the decimation processing on the digital reception signal. In addition, the beam forming block 54 executes the phase alignment and addition processing on the digital reception signal to form the reception beam signal. Thereafter, the processing is sequentially executed in the circuit from the signal processing unit 26.

Although the ultrasound diagnostic apparatus according to the present disclosure has been described above, the ultrasound diagnostic apparatus according to the present disclosure is not limited to the above-described embodiment, and various changes can be made without departing from the gist thereof.

In the present embodiment, each processing is executed by any computer. In addition, any computer may execute these types of processing by a processor as hardware, a program as software, or a combination thereof. In such a case, the processor is configured to execute various types of processing in the present embodiment in cooperation with the program, and may function as each unit or each means in the present embodiment. In addition, the execution order of the processing by the processor is not limited to the above-described order and may be changed as appropriate. Any computer may be a general-purpose computer, a computer for specific use, a workstation, or another system capable of executing each processing.

The processor may be composed of one or a plurality of pieces of hardware, and types of hardware are not limited. For example, the processor may be composed of hardware such as a central processing unit (CPU), a micro processing unit (MPU), a programmable logic device such as a field programmable gate array (FPGA), a dedicated circuit for executing specific processing, such as an application specific integrated circuit (ASIC), a graphic processing unit (GPU), or a neural processing unit (NPU). Further, the type of hardware may be a combination of different types of hardware. In a case in which the plurality of types of hardware are configured to execute one or a plurality of types of processing of a certain processor, the plurality of types of hardware may exist in devices physically separated from each other or may exist in the same device. Furthermore, in any of the embodiments, the order of each processing performed by the processor is not limited to the above-described order, and may be changed as appropriate. The hardware is composed of an electric circuit (circuitry) in which circuit elements such as semiconductor elements are combined.

The program may be software such as firmware or a microcode. Furthermore, the program may be, for example, a program module group, and each function thereof may be implemented by a processor configured to execute each function. The program may be a program code or a plurality of code segments stored in one or a plurality of non-transitory computer-readable media (for example, a storage medium and other storages). The program may be stored in the plurality of non-transitory computer-readable media existing in physically separated devices. The program code or the code segment may represent any combination of procedures, functions, subprograms, routines, subroutines, modules, software packages, classes, instructions, data structures, or program statements. The program code or the code segments may be connected to other code segments or hardware circuits by transmitting and receiving information, data, an argument, a parameter, or content of a memory.

The present invention can also be applied to a program and a program product.

What is claimed is:

1. An ultrasound diagnostic apparatus comprising:
an ultrasound probe that transmits an ultrasound wave to a subject and receives a reflection of the ultrasound wave from the subject to output an analog reception signal;
a reception circuit that processes the analog reception signal; and
a processor,
wherein the processor is configured to:
identify a type of the ultrasound probe;
control the reception circuit according to the type of the ultrasound probe; and
change at least a part of a circuit configuration of the reception circuit.

2. The ultrasound diagnostic apparatus according to claim 1,
wherein the reception circuit includes:
an AD converter that converts the analog reception signal into a digital reception signal; and
a processing circuit that processes the digital reception signal, and
the processor is configured to:
control the AD converter such that the AD converter converts the analog reception signal into the digital reception signal at a sampling frequency corresponding to the type of the ultrasound probe; and
change a circuit configuration of the processing circuit according to the type of the ultrasound probe.

3. The ultrasound diagnostic apparatus according to claim 2,
wherein the processing circuit executes decimation processing of the digital reception signal.

4. The ultrasound diagnostic apparatus according to claim 3,
wherein the processor is configured to change a circuit configuration of a filter for the decimation processing according to the type of the ultrasound probe.

5. The ultrasound diagnostic apparatus according to claim 4,
wherein the processor is configured to:
control the AD converter such that the AD converter converts the analog reception signal into the digital reception signal at a higher sampling frequency as a frequency of the analog reception signal output by the ultrasound probe increases; and
reduce a number of filters in accordance with the type of the ultrasound probe.

6. The ultrasound diagnostic apparatus according to claim 2,
wherein the processing circuit is implemented by an FPGA.

7. The ultrasound diagnostic apparatus according to claim 6,
wherein the processor is configured to change a part of a circuit configuration of the FPGA.

8. The ultrasound diagnostic apparatus according to claim 1,
wherein the processor is configured to notify an operator of the ultrasound diagnostic apparatus whether the change of the circuit configuration of the reception circuit is successful or unsuccessful.

9. A non-transitory computer-readable storage medium storing an ultrasound diagnostic program causing an ultrasound diagnostic apparatus to execute:
identifying a type of an ultrasound probe that is connected to the ultrasound diagnostic apparatus, and that transmits an ultrasound wave to a subject and receives a reflection of the ultrasound wave from the subject to output an analog reception signal; and
controlling a reception circuit that processes the analog reception signal according to the type of the ultrasound probe and changing at least a part of a circuit configuration of the reception circuit.
